(19) United States
(12) Patent Application Publication
(10) Pub. No.: US 2024/0350335 A1
(43) Pub. Date: Oct. 24, 2024

(54) FLUID COLLECTION HANGER AND METHOD OF HANGING A FLUID COLLECTION BAG ON A WHEELCHAIR

(71) Applicant: HERMAN EAVES, VINCENNES, IN (US)

(72) Inventor: HERMAN EAVES, VINCENNES, IN (US)

(21) Appl. No.: 18/643,803

(22) Filed: Apr. 23, 2024

Related U.S. Application Data
(60) Provisional application No. 63/497,768, filed on Apr. 24, 2023.

(51) Int. Cl. A61G 5/10; A61F 5/44; A61F 5/451

(52) U.S. Cl. CPC A61G 5/10; A61F 5/4404; A61F 5/451

(57) ABSTRACT

A fluid collection bag hanger and method of hanging a fluid collection bag on a wheelchair are provided. The hanger includes a first end configured to attach to the folding frame, and a longitudinally extending arm extending from the first end of the bag hanger to a second end of the bag hanger, which includes a hook for hanging the fluid bag. The method includes placing the folding frame wheelchair in a partially folded position, inserting a first end of the fluid collection bag hanger between the seat bar and upper side arm, moving the seat bar and upper side arm toward one another such that the upper opening engages the upper side arm and the lower opening engages the seat bar, and hanging a fluid collection bag from the hook at the second end of the fluid collection bag hanger.

### BACKGROUND OF THE INVENTION

### Field of the Invention

The present invention relates to systems and methods for collecting bodily fluid in a medical setting. More specifically, the present invention relates to a system and method for collecting bodily fluid from a patient while the patient is seated in a wheelchair.

### Description of the Related Art

Fluid collection systems are typically used in hospitals to monitor the discharge of urine, blood, or exudate from patients with mobility concerns. Such fluid collection systems include a fluid collection bag. A catheter is typically connected to the fluid collection bag and is generally suspended from some object near the patient. Fluid collection bags generally have an inlet tube near the uppermost portion of the bag where the urine enters and an outlet near the bottom portion of the fluid collection bag, which permits discharge of the urine.

When a patient needs to be moved from a medical bed to another location, often times the patient is moved using a wheelchair. In such instances, the fluid collection system typically needs to travel with the patient. Presently, when a patient that is connected to a fluid collection system needs to be moved in a wheelchair, the collection bag is removed from the hanger on the medical bed and is temporarily hung from an available crossmember of the wheelchair. Several problems are associated with such an arrangement. The connection of the fluid collection bag to the wheelchair is not secure which can result in the bag falling from the chair and rupturing. The lines connecting the patient to the bag may also become entangled when not properly attached to the wheelchair frame in the appropriate location.

Accordingly, there is a need for a system and method for collecting bodily fluid from a patient while the patient is seated in a wheelchair that provides a safe and secure connection of the collection bag to the wheelchair in a location that will not result in entanglement of the fluid collection lines with elements of the wheelchair or the patient.

### SUMMARY OF THE INVENTION

It is therefore an object of the present invention to provide a fluid collection bag hanger that can be quickly and easily attached to a folding frame wheelchair.

It is also an object of the invention to provide a fluid collection bag hanger that holds a fluid collection bag securely in place on a folding frame wheelchair.

The present invention meets these objects by providing a system and method for collecting bodily fluid from a patient while the patient is seated in a wheelchair including a fluid collection bag hanger that can be quickly and easily attached to the frame of a folding wheelchair and positions the bag so that it is securely hung from a location that will minimize the risk of entanglement of the fluid collection lines.

According to one presently preferred embodiment of the invention, there is provided a fluid collection bag hanger for use on a folding frame wheelchair comprising a first end configured to attach to the folding frame, and a longitudinally extending arm extending from the first end of the bag hanger to a second end of the bag hanger. The first end has a lower opening for engaging an upper side arm of the folding frame, and an upper opening for engaging a seat bar of the folding frame. Means for hanging a collection bag at the second end of bag hanger are also provided.

The lower opening may have a shape which corresponds to the outer surface shape of the upper side arm, and the upper opening may have a shape which corresponds to the outer surface shape of the seat bar. The lower opening and upper opening may be arc-shaped and the upper side arm and seat bar may be round. The fluid collection bag hanger may further comprise an extension at the first end of the hanger extending upwardly from and past the upper opening to provide additional support and rigidity.

The longitudinally extending arm may comprise a first segment that extends downwardly at an angle from the first end, and a second segment that extends from the first segment such that, when in use, the second segment is parallel to the seat bar. The second end may comprise a u-shaped opening. The hanger may be formed from a rigid material, preferably a clear acrylic material.

According to a further preferred embodiment of the invention, there is provided a method of attaching a fluid collection bag hanger to a folding frame wheelchair. First, the folding frame wheelchair is placed in a partially folded position where a seat bar of the folding frame wheelchair is raised a distance above an upper side arm of the folding frame wheelchair. A first end of the fluid collection bag hanger is then inserted between the seat bar and upper side arm such that an upper opening in the first end is aligned with the seat bar, and a lower opening in the first end is aligned with the upper side arm. The fluid collection bag hanger further includes a longitudinally extending arm extending from the first end of the bag hanger toward the center of the wheelchair to a second end of the bag hanger and means for hanging a collection bag at the second end of bag hanger. Next, the seat bar and upper side arm are moved toward one another such that the upper opening engages the upper side arm and the lower opening engages the seat bar. Finally, a fluid collection bag is hung from the hanging means at the second end of the fluid collection bag hanger.

These and other objects, features and advantages of the present invention will become apparent from a review of the following drawings and detailed description of the preferred embodiments of the invention.

### BRIEF DESCRIPTION OF THE DRAWINGS

The present invention can best be understood in connection with the accompanying drawings. It is noted that the invention is not limited to the precise embodiments shown in the drawings, in which:

FIG. 1 is a front perspective view of a folding wheelchair frame as is known in the prior art.

FIG. 2 is a front perspective view of a fluid collection bag hanger according to one presently preferred embodiment of the invention.

FIG. 3 is a top plan view of the fluid collection bag hanger shown in FIG. 2.

FIG. 4 is a front view of the fluid collection bag hanger shown in FIG. 2 and FIG. 3 partially installed on a folding wheelchair frame.

FIG. 5 is a front perspective view of the fluid collection bag hanger shown in FIG. 2 and FIG. 3 partially installed on a folding wheelchair frame.

FIG. 6 is a front perspective view of the fluid collection bag hanger shown in FIG. 2 and FIG. 3 fully installed on a folding wheelchair frame with a fluid collection bag hanging therefrom.

### DETAILED DESCRIPTION OF THE INVENTION

For the purposes of promoting an understanding of the principles of the embodiments described herein, reference is now made to the drawings and descriptions in the following written specification. No limitation to the scope of the subject matter is intended by the references. This disclosure also includes any alterations and modifications to the illustrated embodiments and includes further applications of the principles of the described embodiments as would normally occur to one skilled in the art to which this document pertains.

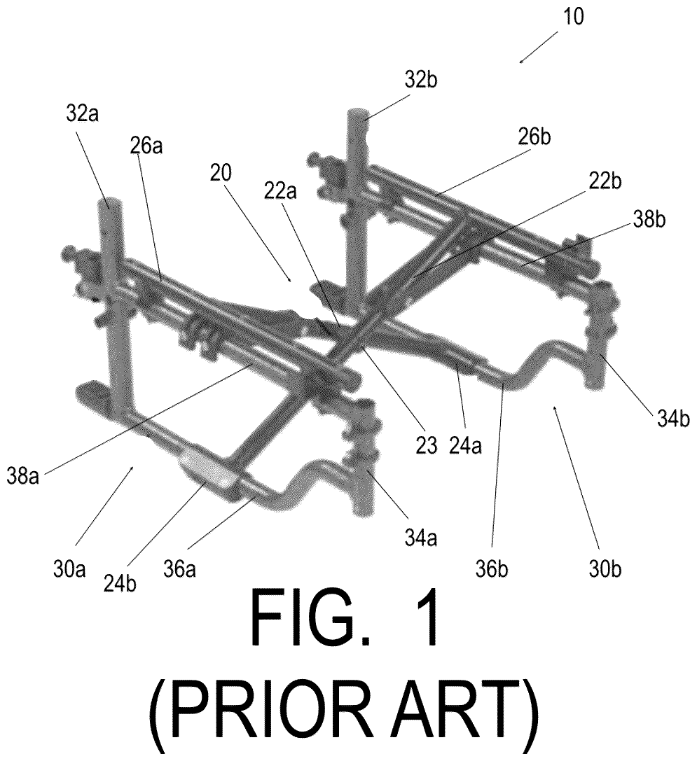

Folding frames wheelchairs are the most common types of wheelchair frame available today because of their ability to fold for transportation and storage. Standard, light weight and light weight adjustable wheelchairs are constructed with folding frames. The seat upholstery on a folding frame is attached to the frame making the wheelchair frame also the seat frame FIG. 1 shows a conventional lightweight folding wheelchair frame 10 as is known in the prior art, and consists of a cross frame 20 and two side frames 30a, 30b. Each side frame 30a, 30b includes a back post 32a, 32b, connected to a front post 34a, 34b by a lower side arm 36a, 36b and an upper side arm 38a, 38b. The cross frame 20 includes first and second cross members 22a, 22b that are pivotally connected to one another at a pivot point 23 near the midpoint of each. A bracket 24a, 24b is provided at the first end of each cross member 22a, 22b to fixedly connect the first ends of the cross members 22a, 22b to the lower side arms 36a, 36b, respectively, of the side frames 30a, 30b. A seat bar 26a, 26b that extends parallel to and above each upper side arm 38a, 38b is attached to the second ends of each cross member 22a, 22b. The seat (not shown), which is typically formed of a flexible fabric material is attached to each of the seat bars 26a, 26b and spans the space therebetween.

FIG. 1 shows the folding wheelchair frame 10 in the open, or in-use position. To close or collapse the wheelchair 10, the seat bars 26a, 26b are raised up causing the cross members 22a, 22b to pivot about pivot point 23. This causes the brackets 24a, 24b attached to the first ends of the cross members 22a, 22b to pull the first and second side frames 30a, 30b toward one another to fold the wheelchair for transportation or storage.

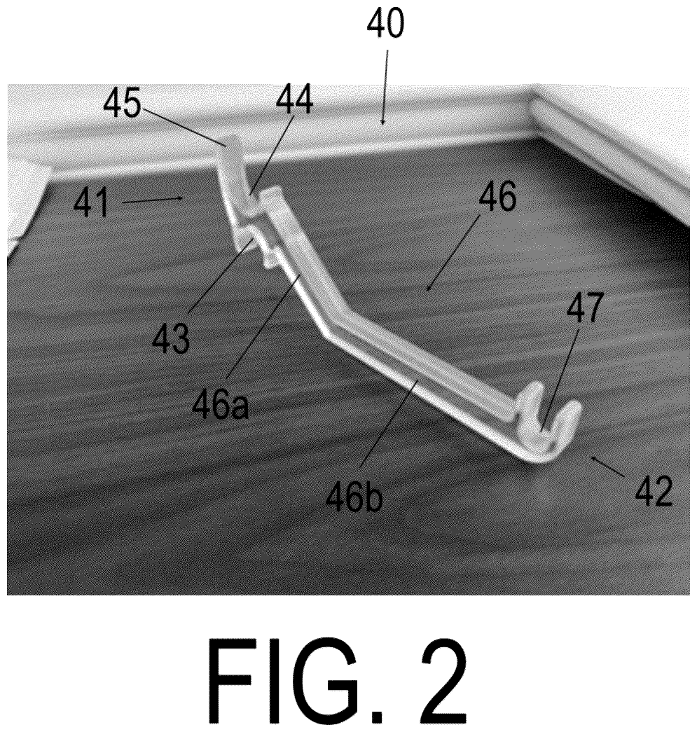

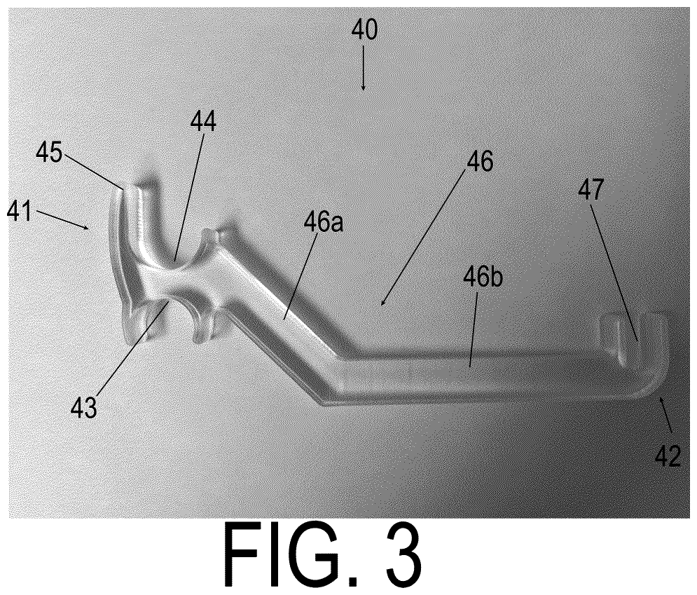

FIG. 2 and FIG. 3 show a fluid collection bag hanger 40 for use on a wheelchair according to a preferred embodiment of the invention. A first end 41 of the fluid collection bag hanger 40 is configured to attach to the frame of a wheelchair frame 10, while a second end 42 is configured for supporting/hanging a fluid bag. For use in connection with the folding wheelchair frames 10 of the type shown in FIG. 1, the first end of the fluid collection bag hanger 40 is provided with a lower opening 43 which is configured to frictionally engage the surface of one of the upper side arms 38a, 38b of one of the side frames 30a, 30b of the folding wheelchair 10. The first end 41 of the fluid collection bag hanger 40 is also provided with an upper opening 44 which is configured to frictionally engage the surface of one of the seat bars 26a, 26b of the cross frame 20. As shown in FIG. 3, the lower opening 43 and upper opening 44 are arc-shaped to correspond to the round shape of the upper side arms 38a, 38b and seat bars 26a, 26b. If the side arms 38a, 38b and seat bars 26a, 26b are not round, the shapes lower opening 43 and upper opening 44 can be modified to fit the shape and size of the side arms 38a, 38b and seat bars 26a, 26b. The upper opening 44 also may include an extension 45 at the first end of the hanger 40 to provide additional support and rigidity.

The first end 41 and second end 42 of the fluid collection bag hanger 40 are connected by a longitudinally extending arm 46. As shown in FIG. 3, the arm 46 may include a first segment 46a that extends downwardly at an angle from the first end 41 of the fluid collection bag hanger 40 and a second segment 46b that extends substantially parallel to seat bars 26a, and side arms 38a, 38b when installed. The second end 42 of the hanger 40 may be provided with a hook or u-shaped opening 47 from which a fluid collection bag may be hung.

The fluid collection bag hanger 40 may be formed of any type of rigid, or semi-rigid material, so long as the material is of sufficient strength to support a filled fluid bag suspended from the second end 42 thereof. According to one presently preferred embodiment of the invention, the hanger 40 is formed from clear acrylic material with a thickness of approximately ½ inch.

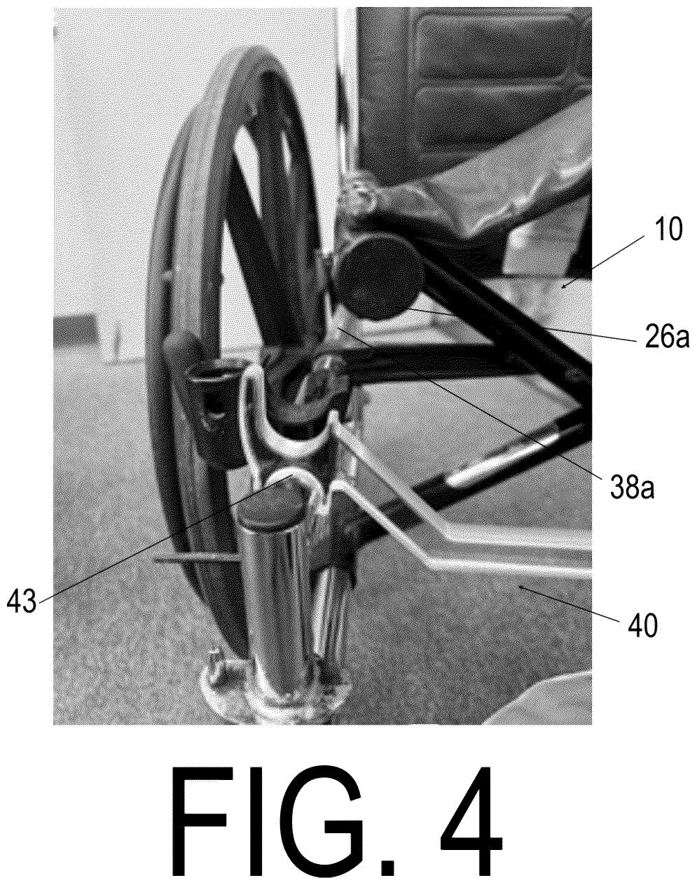

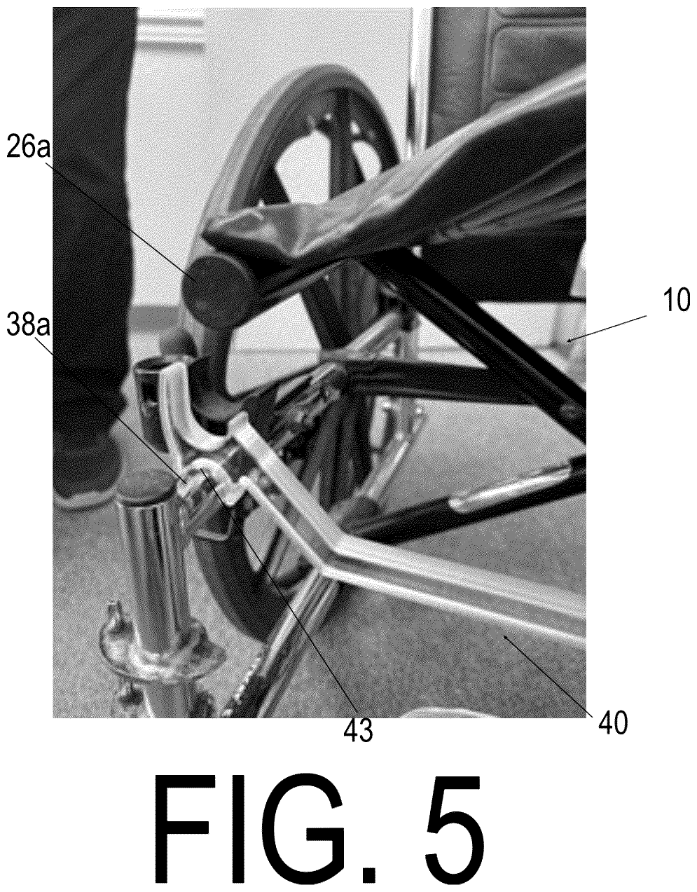

FIG. 4-FIG. 6 show the steps involved in the method of hanging a fluid collection bag on a wheelchair according to one presently preferred embodiment of the invention. As shown in FIG. 4 and FIG. 5, the wheelchair frame 10 is first placed in the folded, or partially folded position where the seat bar 26a, is raised a distance above the upper side arm 38a. The lower opening 43 of the fluid collection bag hanger 40 may then be placed onto the upper side arm 38a. As mentioned previously, the shape and dimensions of the lower opening are such that a friction fit is achieved upon engagement of the lower opening 43 with the upper side arm 38a.

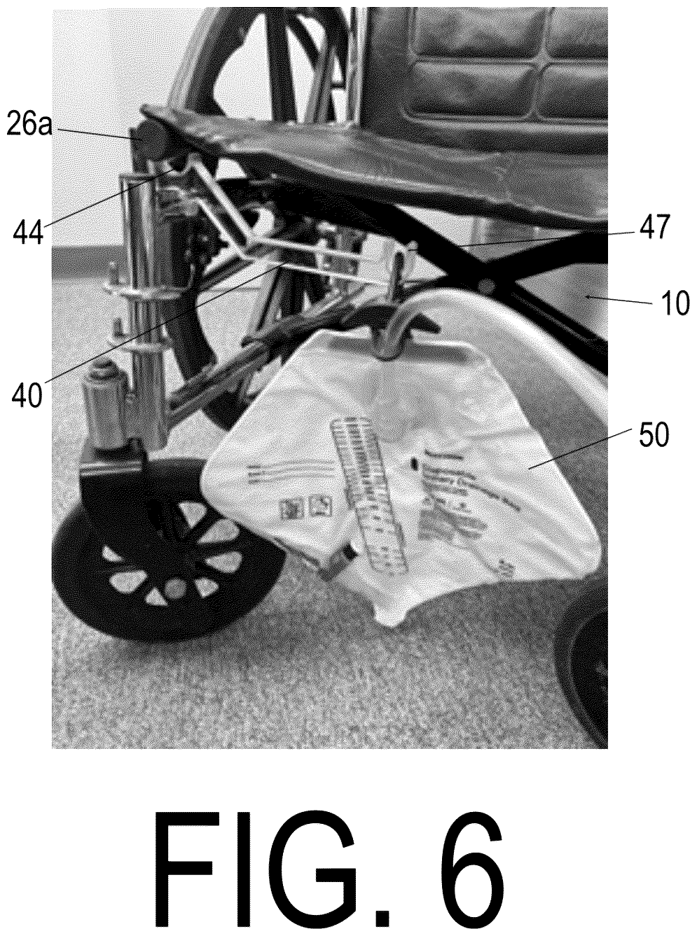

As shown in FIG. 6, the seat bar 26a may then be lowered into engagement with the upper opening 44 of the hanger 40 such that the wheelchair frame 10 is now in the fully open and operable position. A fluid collection bag 50 may then be hung from the hook or u-shaped opening 47 at the second end 42 of the hanger 40.

This detailed description, and particularly the specific details of the exemplary embodiment disclosed, is given primarily for clearness of understanding and no unnecessary limitations are to be understood therefrom, for modifications will become evident to those skilled in the art upon reading this disclosure and may be made without departing from the spirit or scope of the claimed invention.

I/We claim:

1. A fluid collection bag hanger for use on a folding frame wheelchair comprising:
a. a first end configured to attach to the folding frame, said first end having a lower opening for engaging an upper side arm of the folding frame, and an upper opening for engaging a seat bar of the folding frame;
b. a longitudinally extending arm extending from the first end of the bag hanger to a second end of the bag hanger; and
c. means for hanging a collection bag at the second end of bag hanger.

2. The fluid collection bag hanger according to claim 1, wherein the lower opening has a shape which corresponds to the outer surface shape of the upper side arm, and the upper opening has a shape which corresponds to the outer surface shape of the seat bar.

3. The fluid collection bag hanger according to claim 2, wherein the lower opening and upper opening are arc-shaped and the upper side arm and seat bar are round.

4. The fluid collection bag hanger according to claim 1, further comprising an extension at the first end of the hanger extending upwardly from and past the upper opening to provide additional support and rigidity.

5. The fluid collection bag hanger according to claim 1, wherein the longitudinally extending arm comprises a first segment that extends downwardly at an angle from the first end, and a second segment that extends from the first segment such that, when in use, the second segment is parallel to the seat bar.

6. The fluid collection bag hanger according to claim 1, wherein the second end comprises a u-shaped opening.

7. The fluid collection bag hanger according to claim 1, wherein the hanger is formed from a rigid material.

8. The fluid collection bag hanger according to claim 7, wherein the rigid material is a clear acrylic material.

9. A method of attaching a fluid collection bag hanger to a folding frame wheelchair comprising the steps of:
a. placing the folding frame wheelchair in a partially folded position where a seat bar of the folding frame wheelchair is raised a distance above an upper side arm of the folding frame wheelchair;
b. inserting a first end of the fluid collection bag hanger between the seat bar and upper side arm such that an upper opening in the first end is aligned with the seat bar, and a lower opening in the first end is aligned with the upper side arm, wherein the fluid collection bag hanger further includes a longitudinally extending arm extending from the first end of the bag hanger toward the center of the wheelchair to a second end of the bag hanger, and means for hanging a collection bag at the second end of bag hanger;
c. moving the seat bar and upper side arm toward one another such that the upper opening engages the upper side arm and the lower opening engages the seat bar;
d. hanging a fluid collection bag from the hanging means at the second end of the fluid collection bag hanger.

10. The method of attaching a fluid collection bag hanger to a folding frame wheelchair according to claim 9, wherein the lower opening has a shape which corresponds to the outer surface shape of the upper side arm, and the upper opening has a shape which corresponds to the outer surface shape of the seat bar such that the surfaces frictionally engage when the seat bar and upper side arm are moved toward one another.

11. The method of attaching a fluid collection bag hanger to a folding frame wheelchair according to claim 10, wherein the lower opening and upper opening are arc-shaped and the upper side arm and seat bar are round.

12. The method of attaching a fluid collection bag hanger to a folding frame wheelchair according to claim 1, further comprising an extension at the first end of the hanger extending upwardly from and past the upper opening to provide additional support and rigidity.

13. The method of attaching a fluid collection bag hanger to a folding frame wheelchair according to claim 1, wherein the longitudinally extending arm comprises a first segment that extends downwardly at an angle from the first end, and a second segment that extends from the first segment such that, when in use, the second segment is parallel to the seat bar.

14. The method of attaching a fluid collection bag hanger to a folding frame wheelchair according to claim 1, wherein the second end comprises a u-shaped opening from which the fluid collection bag is hung.
